(19) United States
(12) Patent Application Publication
(10) Pub. No.: US 2015/0305621 A1
(43) Pub. Date: Oct. 29, 2015

(54) ARTHRITIS DIAGNOSTIC DEVICE, SYSTEM, AND METHOD OF USING

(71) Applicants: Mohammad Faisal Khan, Edmond, OK (US); Harold D. Curtis, Oklahoma City, OK (US)

(72) Inventors: Mohammad Faisal Khan, Edmond, OK (US); Harold D. Curtis, Oklahoma City, OK (US)

(21) Appl. No.: 14/540,211

(22) Filed: Nov. 13, 2014

Related U.S. Application Data
(60) Provisional application No. 61/995,930, filed on Apr. 25, 2014.

(51) Int. Cl. A61B 5/00

(52) U.S. Cl. CPC A61B 5/0053; A61B 5/0002; A61B 5/4528; A61B 5/6806; A61B 5/742; A61B 5/7405

(57) ABSTRACT

A medical device for measuring joint force comprises a sleeve configured to receive a User's thumb and having connected thereto a force sensor, control circuitry, and a data transmission module. The force sensor is positioned in the sleeve to measure a force or pressure exerted by the User's thumb on a patient's body when the sleeve is about the User's thumb. The force sensor outputs an analog signal representing the force measured by the sensor. The control circuitry is in electrical communication with the force sensor and includes an analog to digital converter to convert the analog signal to a digital force signal. The data transmission module transmits the digital force signal to a computing device to generate a medical record for the patient.

### INCORPORATION BY REFERENCE STATEMENT

This application claims priority to U.S. Provisional Application No. 61/995,930 filed on Apr. 25, 2014, which is hereby expressly incorporated herein by reference in its entirety.

### BACKGROUND OF THE INVENTIVE CONCEPTS

1. Field of the Inventive Concepts

The inventive concepts disclosed and claimed herein relate generally to arthritis monitoring devices and methods, and more particularly, but not by way of limitation, to devices, systems, software, and methods for measuring, recording and monitoring joint tenderness in a patient.

2. Brief Description of Related Art

Currently rheumatologists perform joint assessment for tender joints by determining the sensitivity of a specific joint to an applied force or pressure. This joint analysis is the primary method used to evaluate a tender joint. The analysis is accomplished by digital palpation wherein a physician manually exerts pressure upon a tender joint by squeezing the joint with his or her thumb and one or two index fingers to elicit a response from the patient. The industry presently quantifies the force applied by presuming the maximum pressure exerted is 4 kg/cm². The physician currently must apply thumb pressure until the thumbnail blanches, and this blanching is used to indicate that roughly 4 kg/cm² pressure is being applied. Due to a variety of factors, this method results in inconsistent evaluation of the amount of force applied to the joint. This inconsistency from physician to physician, and even within the same physician measurements over time, is a major deterrent to establishing a baseline examination procedure for the industry.

During examination, as the force is progressively increased, the patient is asked to respond when he experiences tenderness. The physician then interprets this as an indication of whether the joint is tender or not. Currently the physician must administer the test and transpose the results manually, which is both inconvenient and inefficient. The process is also inaccurate as different physicians will typically squeeze with a different force and thereby cause inconsistency in the evaluation process. The inconsistency makes establishment of any baseline questionable; however, pharmaceutical companies testing new treatment options would benefit from an established baseline from which to test the validity of the new treatments.

There are currently force and pressure sensors available commercially and utilized in the industrial and medical arenas. However, the force sensors are not utilized to record or measure the force exerted during evaluation for ongoing treatment options. In view of the foregoing, there is a need for a device that can measure the force or pressure applied to a joint by a medical professional. It is to such a device that the presently disclosed and claimed inventive concepts are directed.

### SUMMARY OF THE INVENTIVE CONCEPTS

The inventive concepts disclosed and claimed herein generally relate to apparatuses, systems and methods for measuring force applied to a joint during assessment of the joint. In one embodiment, a medical device for measuring joint force comprises a sleeve configured to receive a User's thumb. A force sensor is attached to the sleeve and is adapted to measure a force or pressure exerted by the User's thumb on a patient's body when the sleeve is about the User's thumb. The force sensor outputs an analog signal representing the force measured by the sensor. Control circuitry connected to the sleeve and in electrical communication with the sensor includes an analog to digital converter to convert the analog signal to a digital force signal. A data transmission module, also connected to the sleeve, transmits the digital force signal to a computing device.

In some embodiments, the present disclosure describes a medical device for measuring joint force provided with a sleeve, a force sensor, a data transmission module and control circuitry. The sleeve is configured to receive a user's thumb. The force sensor, the data transmission module and the control circuitry are connected and/or supported by the sleeve. The force sensor is positioned to measure a force exerted by the user's thumb on a patient's body when the sleeve is about the user's thumb, and configured to output a signal representing the force. The data transmission module is configured to transmit a force signal wirelessly to a computing device. The control circuitry is in electrical communication with the force sensor and configured to provide information indicative of the signal to the data transmission module and to enable the data transmission model to transmit the force signal.

In some embodiments, the medical device for measuring joint force is used in combination with a computing device to form a medical record. The computing device includes non-transitory computer readable media storing software that includes instructions that when executed by the processor cause the processor to instruct the User to apply thumb pressure to a specified joint on the patient, to receive and record digital force signals from the data transmission module, the digital force signals corresponding to the force applied to the specified joint, and query the user regarding a presence or absence of tenderness when force was applied to the first specified joint. The instructions may further cause the processor to receive and record a response to the query, and to record the digital force signals that are indicative of the analog signal originating from the sensor.

### BRIEF DESCRIPTION OF THE DRAWINGS

Like reference numerals in the figures represent and refer to the same or similar element or function. Implementations of the disclosure may be better understood when consideration is given to the following detailed description thereof. Such description makes reference to the annexed pictorial illustrations, schematics, graphs, and drawings. The figures are not necessarily to scale and certain features and certain views of the figures may be shown exaggerated, to scale or in schematic in the interest of clarity and conciseness. In the drawings:

FIG. 1 is an elevational view of one embodiment of a device for measuring force applied to a joint constructed in accordance with the presently disclosed and claimed inventive concepts.

FIG. 2 is an elevational view of another embodiment of a device configured for measuring force applied to a joint constructed in accordance with the presently disclosed and claimed inventive concepts, wherein the device comprises a sheath strapped to a User's hand.

FIG. 3 is an elevational view of yet another embodiment of a device configured for measuring force applied to a joint constructed in accordance with the presently disclosed and claimed inventive concepts, wherein the device comprises a sleeve configured as a spring-hinged cuff.

FIG. 4 illustrates a User measuring joint force using a device constructed in accordance with the presently disclosed and claimed inventive concepts, and transmitting information to a computing device.

FIG. 5 illustrates a flow chart of an exemplary method for evaluating a patient according to presently disclosed and claimed inventive concepts.

FIG. 6 illustrates a computer device screen of an iPad requesting measurement of the right temporomandibular, using both written words and a red highlight of the left temporomandibular on a pictorial representation.

FIG. 7 is a partially exploded view of a device configured for measuring force applied to a joint and described in the Example.

### DETAILED DESCRIPTION OF EXEMPLARY EMBODIMENTS

Before explaining at least one embodiment of the inventive concepts disclosed herein in detail, it is to be understood that the inventive concepts are not limited in their application to the details of construction, exemplary data, and/or the arrangement of the components set forth in the following description, or illustrated in the drawings. The presently disclosed and claimed inventive concepts are capable of other embodiments or of being practiced or carried out in various ways. Also, it is to be understood that the phraseology and terminology employed herein is for purpose of description only and should not be regarded as limiting in any way.

In the following detailed description of embodiments of the inventive concepts, numerous specific details are set forth in order to provide a more thorough understanding of the inventive concepts. However, it will be apparent to one of ordinary skill in the art that the inventive concepts within the disclosure may be practiced without these specific details. In other instances, well-known features have not been described in detail to avoid unnecessarily complicating the instant disclosure.

As will be apparent to those of skill in the art upon reading this disclosure, each of the individual embodiments described and illustrated herein has discreet components and features which may be readily separated from the combined with the features of any of other several embodiments without departing from the scope or spirit of the present disclosure. Any recited method can be carried out in the order of events recited wherein any other order that is logically possible.

Embodiments of the present disclosure will employ, unless otherwise indicated, techniques of electronics, electrical engineering, medical technologies, and the like, which are within the skill of the art. Such techniques are explained fully in the literature.

The term “patient” as used herein includes humans and other warm-blooded animals, such as mammals, for example, dogs, cats, horses, and cattle, and refers to the person or animal whose joint tenderness or sensitivity is being evaluated. The term “user” refers to the one applying the medical device for measuring joint force to the body of the patient. The User can be the same as the patient, or the User can be another such as, for example, a nurse, physician, or veterinarian. In a common setting, the patient is a human rheumatoid arthritis patient and the User is the patient's physician.

The term “joint force” as used herein and in the appended claims refers to the force applied to a joint of a patient. Because utilizing the inventive concepts described herein allows the area of application to remain constant, the term “force” as used herein is inclusive of “pressure.”

Further, unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the inventive concepts. This description should be read to include one or at least one and the singular also includes the plural unless it is obvious that it is meant otherwise.

Finally, as used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

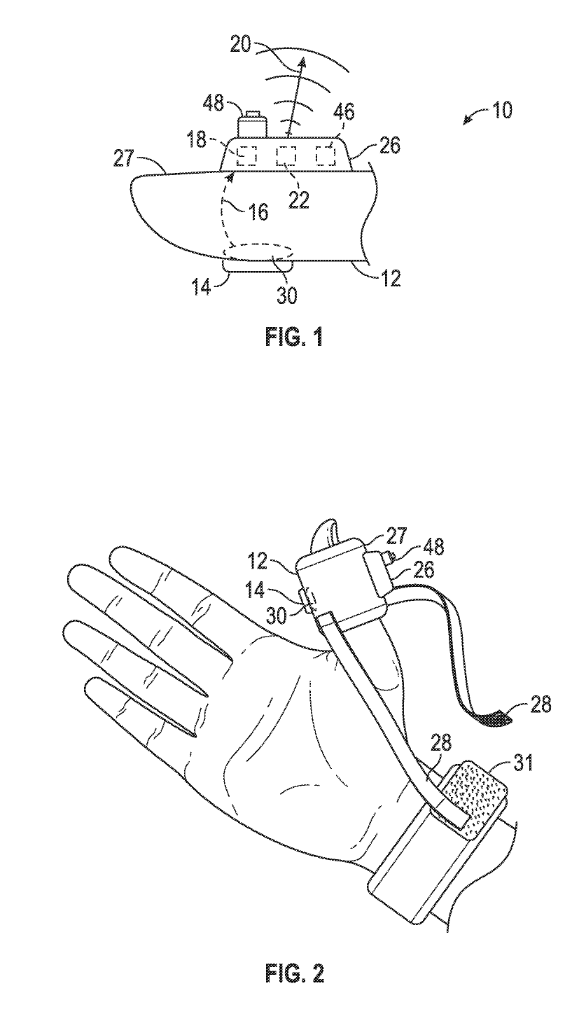

Referring now to the drawings, and more particularly to FIG. 1, shown therein is an exemplary embodiment of a medical device 10 constructed in accordance with the inventive concepts disclosed and claimed herein. In this embodiment, the medical device 10 comprises a sleeve 12 preferably constructed of a flexible material encompassing a space and forming an opening sized and adapted to receive a User's thumb. A force sensor 14 is connected to the sleeve 12 and is adapted to measure a force or pressure exerted by the User's thumb on a patient's body when the sleeve 12 is about the User's thumb. The force sensor 14 may output an analog signal 16 representing the force measured by the sensor 14. Control circuitry 18 connected to the sleeve 12 is in electrical communication with the sensor 14, and may be configured to convert the analog signal 16 to a digital force signal 20. A data transmission module 22 may transmit the digital force signal 20 to a computing device 24 using any suitable communication protocol, such as a protocol within the IEEE 802.11 family, a protocol known in the art as “Bluetooth”, or a cellular telephone protocol, for example.

In some embodiments, the control circuitry 18 and data transmission module 22 are contained in a housing 26 which is attached to the sleeve 12 in a position opposite the sensor 14 as shown in FIG. 1. This can allow the User to more readily interact with the control circuitry 18 as described in detail hereinafter.

The sleeve 12 can be made of a flexible, elastic material such that it can be pulled over the thumb, positioning the housing 26 on a nail side 27 of the thumb, and the sensor 14 on a side opposing the nail at an inside ball 30 of the thumb. The flexible, elastic material allows the sleeve 12 to fit securely about the thumb while accommodating different thumb sizes. In some embodiments, the sleeve 12 can be worn on the User's right thumb or left thumb and can easily be transferred from the left thumb to right thumb so the User, a physician for example, can readily use either hand for examining a joint on the patient.

The sleeve 12 can optionally comprise straps 28 for positioning and securing the housing 26 and the sensor 14 on the User's thumb. The straps 28 can be adjustable in length and can be secured using buckles, snaps, Velcro™, and other such fastening devices known in the art, or can attach to a wrist band 31 using Velcro™ as illustrated in FIG. 2.

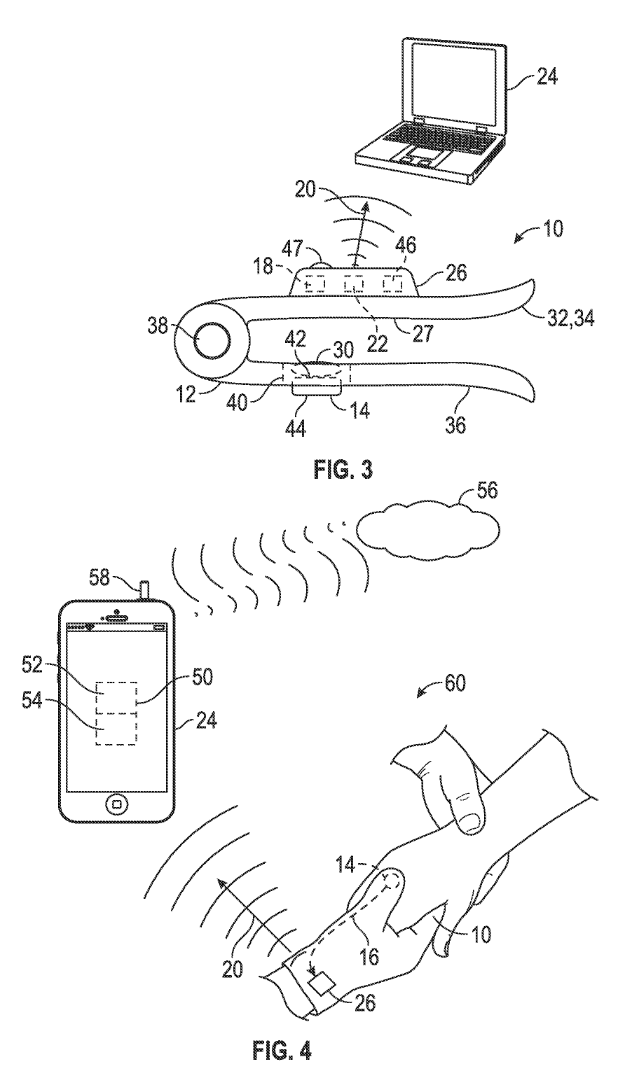

In some embodiments, the sleeve 12 comprises a spring-hinged cuff 32 as shown in FIG. 3. The spring-hinged cuff 32 can accommodate different thumb sizes and can allow the User to readily move the spring-hinged cuff 32 from one of the right or left thumb to the other thumb depending on which provides a more convenient reach for the User to evaluate a specific joint. The spring-hinged cuff 32 can have a first portion 34 and a second portion 36 connected by a spring hinge 38 biased to force the first portion 34 toward the second portion 36. The first and second portions 34 and 36, respectively, can comprise relatively rigid materials such as non-elastic plastics, metals, composites, and the like. The first portion 34 can be shaped and configured to include the housing 26 for encompassing and supporting the control circuitry 18. The second portion 36 can include an opening 40 through which the sensor 14 is inserted.

Regardless of the configuration of the sleeve 12, the sensor 14 is positioned such that a first sensor side 42 of the sensor 14 can be in direct or indirect contact with the inside ball of the User's thumb, while a second sensor side 44 of the sensor 14 can placed in direct or indirect contact with the skin about a patient's joint. The second sensor side 44 can be cushioned such that the force is exerted uniformly over a specific surface area even though the patient's joint area is not uniformly flat over the same surface area. In this manner, the User can press his or her thumb on a joint of a patient and the sensor 14 will measure the force or pressure exerted by the User on the patient's joint. Since the area of force measurement is the same for any user, the pressure applied can be duplicated regardless of the size or shape of the user's thumb. The force measurement can readily be converted to pressure and vice versa. Reference to “force” herein is therefore inclusive of “pressure.”

The “indirect contact” mode presumes the sensor 14 is separated from the User's thumb and/or the patient's skin by a material that does not significantly affect force measurement by the sensor 14. For example, the sensor 14 could be held in place on the sleeve using a thin pocket of material to provide comfort to the User. Similarly, a plastic shield such as commonly used by physicians for sanitary purposes could shield the patient from direct contact with the sensor 14.

There are many force and pressure sensors available and suitable for the present application and known to those skilled in the art. The terms “force sensor” used herein includes devices labeled as a “pressure sensor” as the measurements are proportional to each other and can be used interchangeably. Suitable force sensors include, for example, piezoresistive strain gauges which use strain gauges to detect strain due to applied pressure. Common types of strain gauges are constructed of silicon, polysilicon thin film, bonded metal foil, copper, and combinations thereof, etc. The sensor 14 (such as a strain gauge) can be connected to multiple resistors to form a Wheatstone bridge circuit to maximize the output of the sensor 14 and to reduce sensitivity to errors. For example, FlexiForce® sensors can be purchased covering a range of from 0 to 4 kg force or greater, although other suitable force sensors are readily available. The FlexiForce® sensors utilize a flexible printed circuit with conductive traces that changes resistance in response to application of force.

The control circuitry 18 is in electrical communication with the sensor 14 and may be configured to receive an analog signal from the sensor 14, which can be indicative of the force applied by the User against the patient's joint, and to convert the analog signal to a digital force signal. Analog to digital conversion is well understood by those skilled in the art. Electrical communication can be by wire or any conductive material or surface.

The data transmission module 22 is configured to receive the digital force signals and transmit the digital force signals to the computing device 24. The data transmission module 22 may transmit the digital force signals to the computing device 24 using a wired or wireless transmission system and protocol, such as those known in the art as Bluetooth®. In some embodiments, the control circuitry 18 positioned within the housing 26 further includes a power source 46, such as a battery, for powering the control circuitry 18 and the data transmission module 22.

In some embodiments, the sleeve includes an indicator 47 in communication with the sensor 14 or the control circuitry 18 to alert the User when a target force is being applied to the patient's joint. The indicator 47 can be a visible light such as an LED, an audible indicator such as a buzzer, or other indication means as known to those skilled in the art. For example, as the User increases pressure to the patient's joint, and the sensor 14 reaches a target force of 4 kg, a red LED in the housing 26 and visible to the user can light, alerting the User that the target force or pressure has been reached.

To enable easier communication between the User and the computing device 24, in some embodiments the sleeve 12 additionally includes a response button 48 in electrical connection with the data transmission module 22. The response button 48 can be pushed or otherwise activated by the User to communicate information to the computing device 24. This allows a physician, for example, to communicate a response to the computing device 24 while remaining in immediate contact with the patient being examined. The physician need not move across the room or otherwise leave the patient's side in order to access the computing device 24. In other embodiments, the sensor 14 can be used to indicate a response and communicate information to the computing device 24.

The entire medical device 10 can include only one pressure sensor and can be contained on or within a single sleeve to be worn about only one thumb. In other embodiments and as shown in FIG. 4, the medical device 10 for measuring joint force can take the form of a glove having the sensor 14 in the thumb of the glove while the housing 26 for the control circuitry 18, data transmission module 22, and power source 46 can be located on, for example, the wrist area of the glove. It is important for a physician to have hands free from wires and others elements that distance the physician from the patient or otherwise take the physician's attention away from the patient. The medical device 10 can be used without encumbering the other fingers of the User such as would occur with a glove for use by therapists having a pressure sensor on each finger. Thus, even in a glove form, the entire medical device 10 can include only one pressure sensor contained on or within the thumb portion of the glove.

In some embodiments and as further shown in FIG. 4, the medical device 10 for measuring joint force is used in combination with the computing device 24 having an input device and output device to form a medical record. The computing device 24 is provided with computer hardware 50 including a processor 52 in communication with a non-transitory computer readable medium 54. The non-transitory computer readable medium 54 stores logic, such as software that includes instructions that when executed by the processor 52 cause the processor 52 to record digital force signals originating from the sensor 14. Exemplary non-transitory computer readable medium 54 may include random access memory, read only memory, flash memory, and combinations thereof. The term “non-transitory computer readable medium,” as used herein, includes implementation as a single physical device or multiple physical devices of a distributed system that may or may not be logically related.

The computing device 24 can be implemented as a personal computer, a smart phone, a network-capable TV set, TV set-top box, a tablet, an e-book reader, a laptop computer, a desktop computer, a network-capable handheld device, a video game console, a server, and combinations thereof, for example. Preferably the computing device comprises an input device, an output device, and computer hardware (which is shown in Phantom). The computer hardware may be a system or systems that are able to embody and/or execute the logic of the processes described herein. Logic embodied in the form of software instructions and/or firmware may be executed on any appropriate hardware. For example, logic embodied in the form of software instructions or firmware may be executed on a dedicated system or systems, or on a personal computer system, or on a distributed processing computer system, and/or the like. In some embodiments, logic may be implemented in a stand-alone environment operating on a single computer system and/or logic may be implemented in a networked environment, such as a distributed system using multiple computers and/or processors. The term “processor” as used herein may include a single processor or multiple processors working independently and/or together to execute the logic described herein.

The input device of the computing device 24 is capable of receiving information input from a User, and transmitting such information to the computer hardware. The input device can be implemented as a keyboard, a touchscreen, a mouse, a trackball, a microphone, a fingerprint reader, an infrared port, a slide out keyboard, a flip out keyboard, a cell phone, a PDA, a video game controller, a remote control, a fax machine, and combinations thereof, for example.

The output device of the computing device 24 outputs information in a form perceivable by a User. For example, the output device can be a computer monitor, a screen, a touchscreen, a speaker, a website, a TV set, a smart phone, a PDA, a cell phone, a fax machine, a printer, a laptop computer, and combinations thereof. It is to be understood that the input device and the output device may be implemented as a single device, such as for example a touchscreen of a smart phone or tablet.

The computing device 24 may also include a transceiver for transmitting/receiving information signals to/from a base station or web server 56 via, for example, a cellular antenna 58. Thus, electronics of the computing device 24 can be used to store information from the medical device 10, and/or transmit the information to a base station or a specific communication address via wireless and/or wired, and/or optical communication technology well understood by those skilled in the art.

A system 60 for collecting diagnostic information of a patient is exemplified in FIG. 4 and can include the medical device 10 for measuring joint force as described above, and the computing device 24 having the non-transitory computer readable medium 54 storing software that includes instructions that when executed by the processor 52 cause the processor to instruct a User having the medical device 10 on the User's thumb to apply thumb pressure to a specified joint on a patient. The processor 52 receives and records the digital force signals 20 from the data transmission module 22, the digital force signals 20 corresponding to the force applied to the specified joint by the user. The processor 52 then queries the User regarding a presence or absence of tenderness when force is applied to the specified joint, and receives and records the User's response to the query.

In some embodiments, a predetermined series of joints are to be evaluated. The software can include instructions that when executed by a processor cause the processor to instruct the User (having the medical device on the user's thumb) to sequentially apply thumb pressure to each joint in the predetermined series of joints on the patient. The processor may further be instructed to receive and record digital force signals corresponding to each of the joints in the predetermined series and to sequentially query the User regarding a presence or absence of tenderness when force is applied to each of the joints in the predetermined series. The processor may be instructed to receive and record a response to each of the queries, thereby creating a medical record for the patient. This medical record can readily be duplicated by a second User examining the same patient and using the same or different type of medical device 10, because each User will know when the User is exerting the “standard” 4 kg force on the joint examined, or another agreed-upon level of force. This is a significant advantage over present practices wherein the physician must subjectively evaluate the level of force as to the force is being applied.

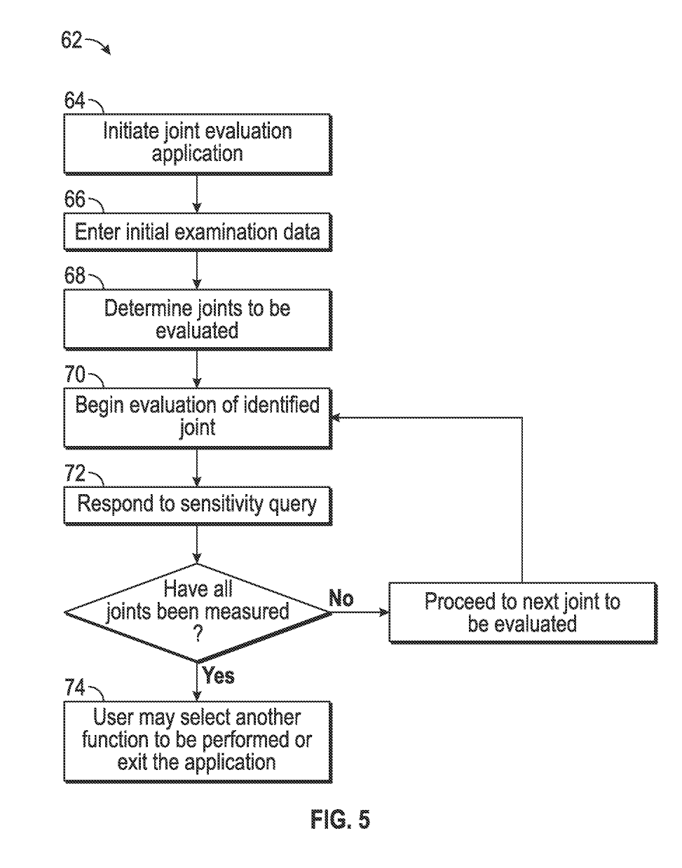

Methods for testing an arthritis patient utilizing the medical device 10 and the computing device 24 include sequentially measuring joint force on joints of the patient. FIG. 5 illustrates a flow chart 62 of an exemplary method for evaluating the patient. The User can initiate a joint examination of the patient as indicated by a step 64. The User may then enter initial examination data to the computing device 24 in a step 66. The initial examination data may include such information as the patient's name, the User's (physician's) name, examination date, patient medications and time of last medication, and the like. The User can then determine the joints to be examined as indicated at a step 68. The set of joints to be evaluated may be selected from an existing joint set stored or obtained by the computing device 24, or a new joint set can be created with input from the user via the input device based on the User's evaluation of the patient, for example.

The physician may begin evaluation of a first identified joint as indicated at step 70. The first joint may be, for example, the left temporomandibular, and the computing device 24 may identify the first joint to be examined using a voice directed system. For example, the processor 52 may direct signals to an output device, e.g., a speaker of the computing device 24 to verbalize “Move to the left temporomandibular.” The physician would first insert his or her thumb into the sleeve 12 and press the sensor 14 against the patient's left temporomandibular until the patient either indicates sensitivity or the computing device indicates that 4 kg force, for example, is being exerted by the physician's thumb against the patient. The processor 52 can record a measurement when the digital force signals 20 indicate the force was increased and was then released. The computing device 24 can then request the physician respond to a sensitivity query as indicated at step 72. The request may be audible, visual, or both. The physician can enter the response using the input device on the computing device 24, or more conveniently by using the response button 48 which can send a signal to the computing device 24 in a wired or wireless fashion, such as by BLUETOOTH®. In another embodiment, the physician can respond to a voice command by quickly pushing on the sensor 14. For example, the computing device 24 may verbalize “Push once for tenderness. Push twice for no tenderness. Hold for three seconds if not evaluated.” The User can then respond by pushing the sensor 14 or the response button 48 the appropriate number of times.

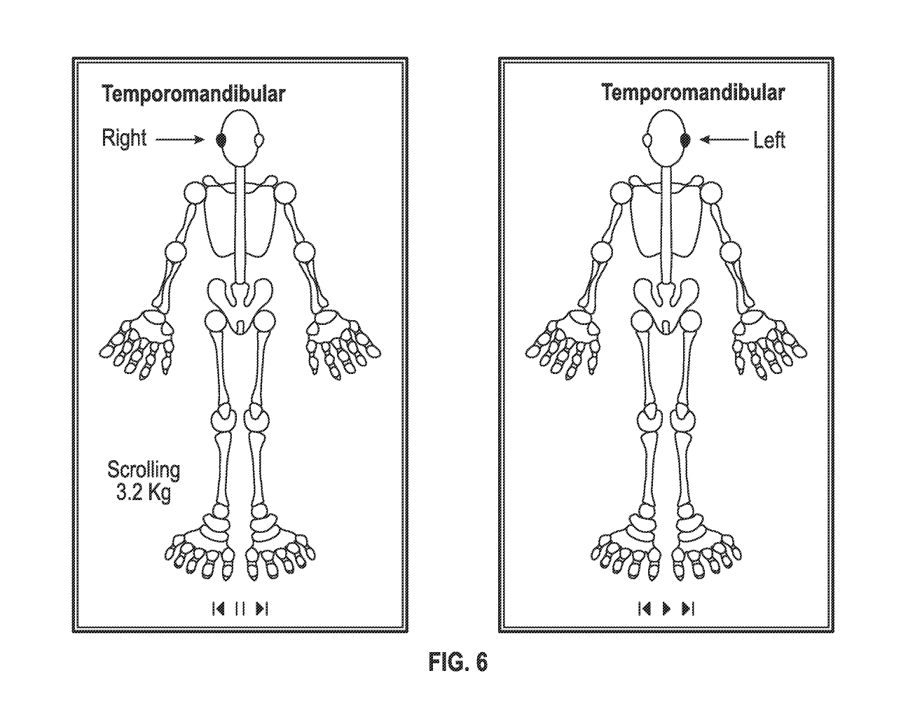

In addition, or alternatively, the output device, e.g., computer screen, of the computing device 24 may show a skeletal diagram as shown in FIG. 6 highlighting the joint to be examined. Additionally, the computer screen may display the force exerted on the joint in real time, so the User will know whether to push harder or whether to reduce thumb pressure.

If all joints have not been measured, step 70 may be repeated on the next sequential joint to be examined. It should be noted that the medical device 10 can readily be changed from the User's left thumb to the User's right thumb when switching examination from one side to the other of the patient. Once the examination is complete, the User may select another function to be performed or exit the application as indicated by step 74.

The device, systems and methods described herein reduce the manual augmentation required of the physician, while improving the accuracy and reproducibility of the measurements. The inventive concepts describe herein may also enable an assistant or nurse to perform the examination process. The data can be utilized and recalled during subsequent patient visits to track the progression and treatment of rheumatoid arthritis in all joints.

### Example

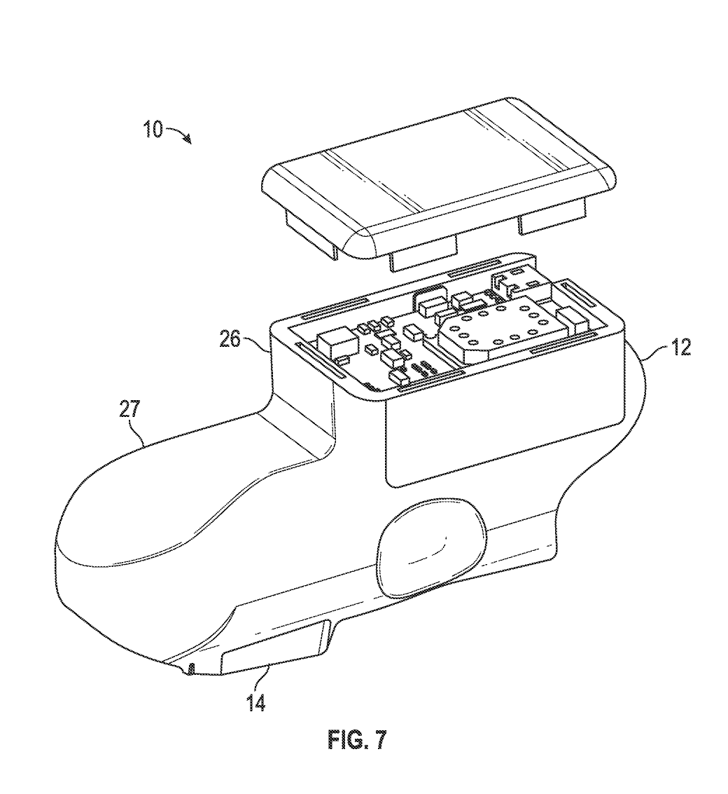

An exemplary rheumatoid arthritis joint assessor, e.g., the medical device 10 comprises a housing built with a soft, flexible plastic that will slip over the User's thumbs with ease and can be assembled so it is an elastic sleeve that provides stability and security to the medical device 10 during the examination process and can be used on either the right or left thumb. The medical device 10, shown in FIG. 7, includes a force sensor positioned so it sets on the ball of the inside of the thumb. The device shown is made of a soft, flexible material. The force is exerted on the patient over a specific and constant surface area such that a given force always results in a specific and known pressure. The area of applied pressure is cushioned on the patient side so that the force is applied uniformly even though the joint area of the patient is non-uniform. This avoids application of the entire force over a relatively small elevated area on the patient. Data from the force sensor is relayed via a processor to a BLUETOOTH® transmitter to communicate with an iPad for the purposes of storing and analyzing the diagnostic testing data performed on the patient. The force sensor or control circuitry can also be connected to a device such as a speaker that produces sound based upon one or more preset force values.

The medical record produced in this manner can be viewed in real-time and can be stored on the iPad or up-loaded to a centralized database or to the general electronic medical files for the patient. This data can be utilized and recalled during subsequent visits to track the progression and treatment of rheumatoid arthritis in one or more or all joints. The medical record can include additional information normally omitted from such joint examinations. For example, if a patient indicates tenderness when the physician exerts only 2 kg force on the examined joint, the physician will typically stop pushing. Thus the record will show not only that the joint was tender, but also that the joint was tender at a 2 kg force level.

From the above description, it is clear that the inventive concepts disclosed herein are well adapted to carry out the objects and to attain the advantages mentioned herein as well as those inherent in the inventive concepts disclosed herein. While exemplary embodiments of the inventive concepts disclosed herein have been described for purposes of this disclosure, it will be understood that numerous changes may be made which will readily suggest themselves to those skilled in the art and which are accomplished without departing from the scope of the inventive concept disclosed herein and defined by the appended claims.

What is claimed is:

1. A medical device for measuring force applied to a joint, comprising:
a sleeve configured to receive a user's thumb, the sleeve having connected thereto;
a force sensor positioned to measure a force exerted by the user's thumb on a patient's body when the sleeve is about the user's thumb, and configured to output a signal representing the force;
a data transmission module configured to transmit a force signal wirelessly to a computing device; and
control circuitry in electrical communication with the force sensor and configured to provide information indicative of the signal to the data transmission module and to enable the data transmission model to transmit the force signal.

2. The medical device of claim 1, wherein the force sensor is positioned between the user's thumb and a cushioned outer area of the sleeve such that the force exerted can be applied evenly over an uneven surface area of the patient.

3. The medical device of claim 1, further comprising a housing connected to the sleeve, the housing dimensioned and positioned to house the control circuitry and the data transmission module.

4. The medical device of claim 1, further comprising an indicator configured to alert the user when a target force is applied to the force sensor.

5. The medical device of claim 1, further comprising a response button connected to the sleeve and configured to allow the user to communicate a response signal to the computing device

6. A system for collecting diagnostic information of a patient, the system comprising:
a medical device including:
a sleeve configured to receive a user's thumb, the sleeve having connected thereto:
a force sensor positioned to measure a force exerted by the user's thumb on a patient's body when the sleeve is about the user's thumb, and configured to output a signal representing the force;
a data transmission module configured to wirelessly transmit a digital force signal based on the signal outputted by the force sensor to a computing device; and
a non-transitory computer-readable storage medium storing software that includes instructions that when executed by a processor cause the processor to:
instruct the user having the medical device on the user's thumb to apply thumb force to a specified joint on the patient;
receive and record digital force signals from the data transmission module, the digital force signals corresponding to the force applied to the specified joint;
query the user regarding a presence or absence of tenderness when force was applied to the first specified joint; and
receive and record a response to the query.

7. The system of claim 6, wherein the force sensor is positioned between the user's thumb and a cushioned outer area of the sleeve such that the force exerted can be applied evenly over an uneven surface of the patient.

8. The system of claim 6, wherein the medical device further includes an indicator configured to alert the user when a target force is applied to the force sensor.

9. The system of claim 6, further comprising a response button connected to the sleeve and configured to allow the user to communicate a response signal to the computing device when queried by the computing device.

10. The system of claim 6, further comprising a battery for powering at least the data transmission module, and a charge cable for charging the battery.

11. The system of claim 6, wherein the software further comprises instructions that when executed by the processor cause the processor to instruct the user to respond to the tenderness query by pressing the force sensor.

12. The system of claim 6, wherein the software further comprises instructions that when executed by the processor cause the processor to communicate, in real time, the force applied by the user to the specified joint.

13. The system of claim 6, wherein the software further includes instructions that when executed by a processor cause the processor to:
instruct the user having the medical device on the user's thumb to sequentially apply thumb pressure to each joint in a predetermined series of joints on the patient;
receive and record digital force signals corresponding to each of the joints in the predetermined series;
sequentially query the user regarding a presence or absence of tenderness when force is applied to each of the joints in the predetermined series; and
receive and record a response to each of the queries.

14. A method for testing an arthritis patient, comprising:
placing a sleeve about a user's thumb, the sleeve having a force sensor, and a data transmission module attached thereto, and aligning the force sensor with an inside ball of the thumb, wherein the force sensor functions to output a signal representing a force exerted by the user's thumb, wherein the digital transmission module is configured to transmit a digital force signal based on the signal representing the force to a computing device;
positioning the inside ball of the thumb over a specified joint of a patient such that the force sensor is between the specified joint and the inside ball of the thumb, the specified joint identified by the computer;
using the thumb to apply force to the force sensor and thereby to the specified joint, resulting in a joint-specific digital force signal communicated to the computing device; and
further communicating to the computing device a joint-specific sensitivity response indicating whether the patient experienced sensitivity during the application of force.

15. The method of claim 14, wherein the medical device further comprises a response button configured to allow the user to wirelessly communicate a response signal to the computing device when queried by the computing device, and wherein the step of communicating the joint-specific sensitivity response comprises pressing the response button.

16. The method of claim 14, further comprising the step of saving the joint-specific digital force signal and the joint-specific sensitivity response as a patient medical record.

17. A non-transitory computer-readable storage medium storing software that includes instructions that when executed by a processor cause the processor to:
pair to a medical device having a force sensor to output an analog signal representing a measured force, control circuitry in electrical communication with the force sensor to convert the analog signal to a digital force signal, and a digital transmission module to transmit the digital force signal to a computing device;
receive a patient identity;
retrieve a prior record(s) of the patient, if available;
receive instructions regarding a series of specific joints to be evaluated;
request measurement of a specified joint in the series, the request either displayed visually on a display screen or audibly through one or more speakers, or both;
receive and record joint-specific digital force signals from the paired medical device;
query the patient's sensitivity to the force exerted on the specified joint; and
receive and record a response to the query.

18. The non-transitory computer-readable storage medium of claim 17, wherein the instructions, when executed by the processor, further cause the processor to generate a medical record for the identified patient.

19. A kit for collecting diagnostic information of a patient, the kit comprising:
a sleeve configured to receive a user's thumb, the sleeve having a force sensor disposed therein and adapted to output an analog signal representing a measured force; control circuitry in electrical communication with the force sensor and configured to convert the analog signal to a digital force signal; a data transmission module configured to wirelessly transmit the digital force signal to a computing device; and a battery to power the control circuitry and the data transmission module; and
software downloadable to a computing device, the software including instructions that when executed by a processor of the computing device cause the processor to record digital force signals from the data transmission module.

20. The kit of claim 19, further comprising a charging device for recharging the battery.
